(19) United States
(12) Patent Application Publication
(10) Pub. No.: US 2024/0199690 A1
(43) Pub. Date: Jun. 20, 2024

(54) METHOD FOR PRODUCING PEPTONES COMPRISING PROTEINS AND AMINO ACIDS FROM MUCOUS MEMBRANES OF ANIMALS TO BE SLAUGHTERED, AND THE PEPTONES THEMSELVES

(71) Applicant: CREMER OLEO GmbH & Co. KG, Hamburg (DE)

(72) Inventors: Steffen MAINZER, Hamburg (DE); Heinrich KLEINE KLAUSING, Hamburg (DE)

(21) Appl. No.: 18/555,048

(22) PCT Filed: Feb. 18, 2022
(86) PCT No.: PCT/EP2022/054073

§ 371 Date: Oct. 12, 2023

(30) Foreign Application Priority Data
Apr. 16, 2021 (EP) 21168920.3

(51) Int. Cl. C07K 1/34; C07K 1/30

(52) U.S. Cl. CPC C07K 1/34; C07K 1/306

(57) ABSTRACT

A method having the following steps: (1) providing a composition containing proteins and amino acids from mucous membranes of animals for slaughter, inorganic salts and water, wherein the sum of the mass of proteins and amino acids in the dry mass of said composition is 30 to 70 weight percent and the mass of inorganic salts in the dry mass of said composition is at least 7.5 weight percent; (2) filtering the composition to obtain a filtrate and a filter cake; (3) cooling the filtrate to a temperature in the range of 15° C. to 15° C. to form a precipitate; (4) separating the resulting precipitate to obtain the liquid phase as product. The disclosure further relates to the products of this method.

### CROSS-REFERENCE TO RELATED APPLICATIONS

This application is a National Stage application of International Patent Application No. PCT/EP2022/054073, filed on Feb. 18, 2022, which claims priority to European Patent Application No. 21168920.3, filed on Apr. 16, 2021, each of which is hereby incorporated by reference in its entirety.

### TECHNICAL FIELD

The invention relates to a method for producing a peptone.

### BACKGROUND

Heparin is an active substance that is mainly used in medicine as an anticoagulant. The majority of industrially produced heparin is obtained from the intestinal mucosa of pigs. In a widely used process, a solution comprising ingredients from the intestinal mucosa of pigs is subjected to enzymatic hydrolysis. The heparin contained in the solution is then adsorbed onto a resin, which is then separated from the liquid phase. The remaining liquid phase is a waste product and, in addition to the amino acids and peptides produced during the enzymatic hydrolysis, also contains other constituents of the intestinal mucosa and salts that are introduced into the liquid phase before and/or during execution of the extraction process. These salts are usually sodium bisulphide, which is used to stabilise the solution against microbial growth and spoilage. Other salts may also be included in this liquid phase. The liquid phase cannot be used as such as feed because of its high salt content. In feed, the high salt content leads to digestive disorders in the animals and, as a result, growth losses may occur. The eluate is therefore a waste product, even though it contains valuable amino acids and proteins. It causes considerable costs when it is disposed of. Therefore, this waste material is usually processed into a peptone, which involves, among other things, removing the water and salts. Peptones are mixtures containing the hydrolysates of proteins, i.e. amino acids and peptides.

The peptones obtained in this way contain a high proportion of amino acids and peptides of the intestinal mucosa and can be used as an additive to feed and the like.

The Chinese disclosure CN 106035980 A describes a method in which the eluate of heparin production is subjected to filtration with a microfiltration membrane or ultrafiltration membrane. The permeate is largely freed of water by heating and distillation, whereby most of the salts contained therein crystallise out. The permeate is then combined with the liquid retentate of the microfiltration, and the mixture is dried. The peptone thus obtained, after the addition of auxiliary substances, is used as feed and the like. This method is very costly due to the microfiltration process.

In WO 2012/069513 A1, a method for processing the eluate from heparin production is presented in which first an ultrafiltration is carried out and then the salts are removed by electrodialysis. This method is also very complex and cost-intensive due to these two method steps. So the problem remains of finding a more cost-effective treatment or use for the waste from heparin production following the enzymatic process and other similar waste.

### SUMMARY

The object of the invention is to provide a method with which waste containing large quantities of inorganic salts and proteins and optionally amino acids can be processed cost-effectively, in particular into feed or feed additives. Preferably, the waste in question is waste from the production of heparin. In particular, it is an object of the present invention to provide a method which is capable of producing a peptone from waste materials produced during heparin production using simple and cost-effective method steps. The peptone shall preferably be usable in animal nutrition. For this purpose, the peptone should contain as low a proportion of inorganic salts as possible. Furthermore, the peptone shall preferably contain the highest possible proportion of amino acids and proteins.

The present invention relates to a method for processing compositions containing mixtures of proteins, amino acids and salts, the proteins and amino acids of which are at least partially derived from mucous membranes of animals for slaughter. The method is one that comprises the following steps:
(1) providing a composition containing proteins and amino acids from mucous membranes of animals for slaughter, inorganic salts and water, wherein the sum of the mass of proteins and amino acids in the dry mass of said composition is 30 to 70 weight percent and the mass of inorganic salts in the dry mass of said composition is at least 7.5 weight percent;
(2) filtering the composition to obtain a filtrate and a filter cake;
(3) cooling the filtrate to a temperature in the range of −15° C. to 15° C. to form a precipitate;
(4) separating the resulting precipitate to obtain the liquid phase as the product.

By way of this method, the precipitate separated in step (4) separates a portion of the inorganic salts contained in the provided composition from the proteins and amino acids also contained in the provided composition. The terms “peptide” and “protein” are used hereinafter with identical meanings, that is to say, both terms are used to refer to oligoamino acids and polyamino acids containing two or more amino acid residues. The method according to the invention is very simple and can be carried out cost-effectively and with a high space-time yield. It is thus superior to the prior art. The actual separation of the inorganic salts is carried out by cooling, forming a precipitate and separating it. The method step (2) of filtering the composition used (provided) serves to prepare (process) the composition and only enables the inorganic salts to crystallise out by cooling.

Preliminary tests have shown that it is not possible to directly crystallise the inorganic salts from the compositions used in step (1) by cooling. At the least, this is not possible in a quantity and at a rate necessary for an industrial process. It is assumed that such compositions contain compounds that inhibit crystallisation. These could be polyanions, such as strongly sulphonated polysaccharides found in mucous membranes. Partially degraded proteins from mucous membranes could also have such an effect. Surprisingly, however, it is easily possible to crystallise inorganic salts from these compositions in step (3) with a high space-time yield after separation of the solids by filtration in step (2). Only then does the method according to the invention become commercially viable.

The high proportion of inorganic salts in the composition provided in step (1) prevents the commercial use of the proteins and amino acids contained therein as additives to feed and the like. If such compositions cannot be reprocessed, they must be disposed of at great expense. Here, the costs for reprocessing are decisive for the commercial benefit of the method. All three substances obtained in this method, i.e. the filter cake from step (1), the precipitate separated in step (4) and the liquid phase obtained in step (4), contain proteins and amino acids as well as inorganic salts. However, the precipitate contains a much higher proportion of inorganic salts than the other two substance compositions. The inorganic salts of the precipitate formed in step (3) remove some of the inorganic salts from the composition provided in step (1). Surprisingly, the filter cake separated in step (2) also contains significantly fewer inorganic salts than the composition provided in step (1). As a rule, the proportion of inorganic salts in the dry mass of the filter cake from step (2) and in the dry mass of the liquid phase obtained in step (4) is, independently of one another, at least 25 weight percent lower than the proportion in the dry mass of the inorganic salts in the composition provided in step (1). Preferably, the proportion of inorganic salts in the dry mass of the liquid phase obtained in step (4) is at least 35 weight percent lower, and more preferably at least 40 weight percent lower. Unless otherwise mentioned or apparent from the context, all quantities given in this text refer to the dry mass of the particular substance. For the purposes of the present invention, the proportion of inorganic salts of a substance is determined by measuring the crude ash content.

With the filter cake produced in step (2) and the liquid phase produced in step (4), two directly usable compositions are obtained as method products. They contain significantly fewer inorganic salts than the composition provided and can therefore generally be used without further processing. The filter cake obtained in step (2) and, in particular, the liquid phase obtained in step (4) can be used, for example, in feed mixtures for monogastrics and also in feed mixtures for dogs, cats and fish. Preferably, a total of up to 5 weight percent of these method products is added to the above-mentioned feed mixtures, the amounts here also being calculated on the dry mass of the method products. The method products are very nutritious due to the high protein and amino acid content and are therefore valuable feed additives. Due to the low salt content, there are no undesirable side effects when feeding. The resulting precipitate can usually also be reused. If, for example, it is a precipitate containing predominantly sodium sulphate, it is further used as such, if necessary after a further purification step. The present method is particularly suitable for compositions containing a large proportion of sodium sulphate in the inorganic salts of the compositions provided in step (1).

The method products can be dried, which can be particularly advantageous for transport and storage. The filter cake resulting from step (2) can be dried by any drying method for solids. The liquid phase resulting as the end product can be dried by any method known to a person skilled in the art for drying liquids, but is preferably dried by spray drying.

As a rule, mucous membranes from animals for slaughter cannot be distributed as food. The quantities produced also cannot be fully used as additives to cattle feed, dog food and the like. They are used in the extraction of ingredients such as heparin and other sulphonated polysaccharides and polyaminosaccharides. Mucous membranes from animals for slaughter can also be subjected to hydrolysis of the proteins they contain in order to dispose of these mucous membranes and obtain protein concentrates. The intestinal mucosa of animals for slaughter is generally used for this purpose, since it is produced in large quantities and can be obtained relatively easily. The composition provided in the present method therefore preferably contains proteins and amino acids from the intestinal mucosa of animals for slaughter. Intestinal mucosa from pigs is particularly preferred, as it is available in sufficiently large quantities at low cost. Intestinal mucosa from cattle is preferably not used because of the danger—even if this still only exists theoretically—of bovine spongiform encephalopathy (BSE) occurring in cattle. For the same reason, in step (1) of the method according to the invention, preference is given to compositions which exclusively contain proteins having a mass of less than 10,000 Daltons. In this case, the mass is measured by matrix-assisted laser desorption ionisation with time-of-flight analysis (MALDI-TOF). Particularly preferred are methods according to the invention which are characterised in that the composition provided in step (1) contains proteins and amino acids which are waste products of the extraction of heparin from the intestinal mucosa of animals for slaughter. Furthermore, in step (1) of the method according to the invention, preferably compositions are used which contain proteins and amino acids obtained in a method for producing heparin in which the intestinal mucosa of animals to be slaughtered is subjected to enzymatic hydrolysis. Preferably, other mucous membranes, organs or tissues of animals for slaughter to which salts have been added and which have been subjected to enzymatic hydrolysis are also used.

The filtration of the composition used in step (2) can in principle be carried out using any conventional filtration method. For example, the filtration method can be selected from the group consisting of chamber filter filtration, microfiltration, vacuum filtration, ultrafiltration, and filtration by membrane filter press, with or without post-pressing device. Other filtration methods may also be used, especially those in which filtration is assisted by the application of pressure or vacuum.

Preferably, a microfiltration unit or a chamber filter press is used for the filtration in step (2). The use of a chamber filter press is particularly preferred. The chamber filter press offers many advantages over microfiltration: it requires less maintenance, the acquisition costs are significantly lower and it also has a significantly higher throughput. In addition, the yield of filtration with a chamber filter press is very high. Usually at least 70 weight percent of filtrate is obtained, based on the composition used in step (1), often even at least 80 weight percent. Preferably, filtration is carried out at a temperature in the range of 40 to 80° C.

When using microfiltration, a ceramic microfiltration unit is preferably used. Ceramic microfiltration units are particularly temperature-resistant. In particular, microfiltration can be carried out by dynamic cross-flow filtration with rotating discs. This is preferred. The pore size is preferably in the range of 0.05 to 1 μm. The filtration pressure is preferably in the range of 0.5 to 5 bar. The filtration in step (2) is preferably carried out at a temperature in the range of 30 to 80° ° C.

When using a chamber filter press, any type of chamber filter press can be used. Preferably, a membrane filter press with a post-pressing unit and rinsing function is used. This can improve the yield and increase the content of dry mass in the filter cake. A standard quality can be used for the filter cloths.

For example, vacuum filtration can be carried out with a filter selected from the group consisting of a belt filter, drum filter, disc filter, plate filter and leaf filter.

The composition prepared in step (1) preferably contains 25 to 50 weight percent of proteins and amino acids, 30 to 60, particularly preferably 35 to 50, weight percent of water and/or 7.5 to 30 weight percent of inorganic salts, in each case based on the total mass of the composition. The specification of “25 to 50 weight percent of proteins and amino acids” herein is the indication here the sum of the mass fraction of the proteins and the mass fraction of the amino acids. If a composition contains more than 60, preferably 50, weight percent of water, this water can be removed from it by suitable measures. Therefore, a method according to the invention is preferred, characterised in that providing the composition in step (1) comprises removing water from a starting material. Accordingly, a suitable composition can be prepared from concentrates by adding water. This corresponds to a method according to the invention, characterised in that providing the composition in step (1) comprises adding water to a starting material.

Such compositions can be prepared from wastewaters from heparin production and similar processes by simply removing some of the water. Wastewaters from heparin production usually contain about 80 to 97 weight percent of water. Disposal of this wastewater is extremely expensive because of the large quantity. Because of the large mass, it is also not easy to transport. Manufacturers of heparin must therefore either process the waste themselves or remove some of the water from it to make transport to a waste recycler commercially more attractive. Wastewater from heparin production from which part of the water has been removed and which has the composition directly above is preferred for use in the present method. Such compositions have a viscosity of about 20,000 cPs at 50° C. At 70° C., the viscosity is still about 8500 cPs, which is about the same as the viscosity of honey at room temperature. It is not possible to crystallise inorganic salts from such compositions by conventional methods. Nevertheless, such compositions are still easily filterable. To reduce viscosity, the compositions may be heated during filtration in step (2) of the method according to the invention. Preferably, they are heated to a temperature of 30 to 80° C., particularly preferably to a temperature of 40 to 70° C.

Surprisingly, the filtrate obtained in step (2) from such high-viscosity compositions has a lower viscosity than the compositions provided in step (1). This could favour the crystallisation of inorganic salts from the filtrates obtained in step (2). Particularly preferred is a method according to the invention in which the composition provided in step (1) comprises from 30 to 45 weight percent of proteins and amino acids, from 40 to 50 weight percent of water and/or from 7.5 to 25 weight percent of inorganic salts, in each case based on the total mass of the composition.

The filter cake obtained when filtering, in step (2), the composition provided in step (1) contains proteins, amino acids and inorganic salts, as does the composition itself provided in step (1), but the inorganic salts are present in significantly smaller amounts. Typically, the filter cake contains such small amounts of inorganic salts that it can be used directly as a protein supplement in animal feed or for other purposes. Preferably, the content of inorganic salts of the filter cake obtained in step (2) is less than 25 weight percent based on the dry mass of the filter cake, more preferably less than 20 weight percent. Preferably, the filter cake is washed with water to further reduce the content of inorganic salts. The filter cake can be dried further and can optionally be further processed into a powder by grinding.

Filters with small pore sizes quickly clog during the filtration in step (1). The reason for this is probably that particles with a wide range of sizes are contained in the composition provided in step (1). As a result, the filter pores become clogged with small particles and filtration is impeded. The complications that arise significantly reduce the space-time yield. Therefore, methods according to the invention are preferred, characterised in that a filter with a pore size greater than 2 μm is used to filter the composition, particularly preferably greater than 10 μm. For this reason, it may be preferred not to use microfiltration in step (2).

While it is very difficult or almost impossible to crystallise the inorganic salts contained in the composition used, the inorganic salts in the filtrate obtained in step (2) surprisingly show a clearly pronounced tendency to crystallise. However, in order to bring the inorganic salts to crystallisation in sufficient quantity and at sufficient speed, the filtrate must be cooled. As described above, the filtrate is brought to a temperature in the range of −15 to 15° C. Lower temperatures cause the filtrate to gel or cause water to crystallise out. Both slow down or completely prevent crystallisation of the inorganic salts, and also prevent any precipitate that may have formed from being effectively separated. Higher temperatures result in no crystallisation or only a small amount of precipitate. Preferably, a method according to the invention is characterised in that the filtrate obtained in step (2) is cooled to a temperature in the range of −12.5 to 12.5° C., and particularly preferably in the range of −10° ° C. to 10° C., for the formation of the precipitate in step (3). Most preferred is a range of −5 to 5° C. This results in the greatest possible separation of inorganic salts with a high space-time yield. In the most favourable embodiment, crystallisation is completed in a discontinuous process at temperatures of −5 to 5° C. in about 1.5 to 3 hours. Crystallisation is terminated when further crystals are formed only in small amounts. The most favourable conditions in terms of temperature and time duration can be easily determined by a person skilled in the art by preliminary tests, if necessary taking into account yield and energy expenditure.

Crystallisation can be assisted by conventional methods, for example by adding seed crystals or by introducing energy, for example by stirring, rubbing with a glass rod, etc. Suitable seed crystals are, for example, activated carbon or sodium sulphate crystals, including those obtained in the method according to the invention after drying of the precipitate from step (4) of the method according to the invention. The addition of seed crystals is preferably carried out at a temperature of 5 to 15° C. and the seed crystals are also preferably pre-cooled to a temperature in this range.

### Preparation for Crystallisation

If it turns out that the filtrate obtained according to step (2) has a concentration of inorganic salts that is unfavourable for crystallisation, this concentration can be re-adjusted for the following crystallisation according to step (3) of the method according to the invention. Preference is therefore given to a method according to the invention which is characterised in that, before cooling the filtrate, its water content is brought to a concentration which is favourable for crystallisation. If the concentration of the inorganic salts and thus also the concentration of the proteins and/or amino acids is too high, the filtrate can be diluted with water. If the filtrate has a concentration of inorganic salts that is too low for crystallisation, these can be concentrated by removing water. Preferably, this is done by evaporation of the water. Membrane processes can also be used, but are usually too costly. Whether the concentration of the salts in the filtrate is favourable for crystallisation can easily be checked by preliminary tests. However, if the composition used in step (1) contains the contents of proteins and amino acids, inorganic salts and water mentioned above as preferred, the filtrate has a concentration favourable for crystallisation.

Preferably, the filtrate obtained in step (2) contains 25 to 50 weight percent of proteins and amino acids, 30 to 60 weight percent of water and 10 to 30 weight percent of inorganic salts, in each case based on the total mass of the filtrate. Particularly preferably, the filtrate obtained in step (2) contains 30 to 45 weight percent of proteins and amino acids, 40 to 55 weight percent of water and 10 to 25 weight percent of inorganic salts, in each case based on the total mass of the filtrate.

The precipitate formed in step (4) can be freed from the mother liquor by any known method. However, decantation, centrifugation or filtration are preferred. Since the mother liquor is usually highly viscous, a large amount of mother liquor adheres to the precipitate. It can be removed most thoroughly by centrifugation. Preferred is therefore a method according to the invention which is characterised in that the separation of the precipitate formed in step (3) is carried out by centrifugation in step (4). Most preferred is a combination of decantation and centrifugation or of filtration and centrifugation. This makes it possible to separate the mother liquor from the precipitate easily, quickly and thoroughly.

The (prepared) compositions used in the method according to the invention usually contain sulphate and chloride as anions in larger amounts, with sulphates usually predominating. In addition, sulphites and phosphates are often present in smaller amounts. Sodium and potassium occur predominantly as cations, with sodium again usually being far more common. Divalent cations such as magnesium and/or calcium are also present in minor amounts. The present method can be used for compositions with any inorganic salts. However, it is optimised for the crystallisation of sodium sulphate. This applies in particular to the preferred embodiments. Accordingly, the use (provision) of compositions in step (1) is preferred in which at least a proportion of 50 weight percent of the inorganic salts consists of the elements sodium and sulphur and chloride. Particularly preferred is the use (provision) of compositions in step (1) in which the proportion of sodium sulphate in the total amount of inorganic salts is at least 50 weight percent, and very particularly preferred is at least 70 weight percent.

Accordingly, at least 50 weight percent of the precipitate obtained in step (4) usually also consist of inorganic salts of the elements sodium and sulphur as well as chloride. This means that at least 50 weight percent of the crude ash consist of the sum of the percentages by weight of the elements sodium, sulphur as well as chloride, with the contents of crude ash, sodium, sulphur and chloride being determinable by the measuring methods mentioned below. Preferably, at least 50 weight percent of the precipitate obtained in step (4) consists of sulphates, particularly preferably at least 70 weight percent. This means that at least 50, particularly preferably at least 70, weight percent of the crude ash consists of the sum of the weight percent of the sulphates, with the content of the sulphates being determinable by the measuring method named below. The precipitate also contains proteins, amino acids, water and other salts. The precipitate can be rinsed with a little water or mother liquor. The rinsing water can be discarded or added to the method again.

In the method according to the invention, usually at least two, in particular exactly two products are obtained, namely the filter cake obtained in step (2) and the liquid phase separated in step (4). The products of the method according to the invention are peptones. Preferably, a method according to the invention is characterised in that the liquid phase obtained in step (4) and/or the filter cake obtained in step (2) contains less than 25 weight percent of inorganic salts, more preferably less than 20 weight percent of inorganic salts, most preferably less than 17.5 weight percent of inorganic salts, based on the dry mass. These quantities apply independently to the liquid phase obtained in step (4) and to the filter cake obtained in step (2). The present method reduces the amount of inorganic salts in the products in each case compared to the composition used. Preferably, a method according to the invention is characterised in that the proportion of inorganic salts in the dry mass of the liquid phase obtained in step (4) and/or in the dry mass of the filter cake obtained in step (2) is reduced by at least 25%, preferably by at least 40% and most preferably by at least 45%, compared to the proportion of inorganic salts in the dry mass of the composition used.

Very particularly preferred is a method according to the invention comprising the following steps:
(1) providing a composition containing 30 to 50 weight percent of proteins and amino acids, 30 to 50 weight percent of water and/or 10 to 30 weight percent of inorganic salts;
(2) filtering the composition to obtain a filtrate and a filter cake;
(3) cooling the filtrate to a temperature in the range of −10° C. to 10° C. to form a precipitate;
(4) filtering off the resulting precipitate and recovering the liquid phase as product.

The present invention also relates to a peptone characterised in that it has been produced according to one of the above methods. The present invention also relates to a filter cake which is characterised in that it has been produced according by a method according to the invention.

### EXAMPLES

### Measurement Methods:

Dry substance->Determination of moisture content

### Regulation (EC) 152/2009 Annex III, A; VDLUFA III, 3.1, 2009; ASU F 0001 (EC):2010-09

### Crude Protein

### Regulation (EC) 152/2009 Annex III, C; VDLUFA III, 4.1.1, 1993; ASU F 0003 (EC):2010-09

### Crude Ash

### Regulation (EC) 152/2009 Annex III, M; VDLUFA III, 8.1, 1979; ASU F 0014 (EC): 2010-09

Total sulphur: DIN EN ISO 11885:2009-09

### Sodium: DIN EN ISO 11885:2009-09

### Chloride: DIN EN ISO 10304-1:2009

### Potassium: DIN EN ISO 11885:2009-09

Total phosphorus: DIN EN ISO 11885:2009-09

### Sulphate Analogue DIN 38405-5-2

### Example 1: Filtration by Means of a Chamber Filter Press

A chamber filter press with plate dimensions of 250 mm by 250 mm is used. The chamber filter press is equipped with 21 chambers, which have a total volume of 20.9 L. The filter cloth used is a polypropylene fabric (PP2436) with a mesh size of 1-5 μm. In total, the chamber filter press has a filter surface of approx. 2 m².

Filtration was carried out at 60° C. 91 L of a prepared composition was filtered and 70 L of filtrate was obtained. This corresponds to a yield of 77% by volume. The filter chambers were completely filled. Thus, the amount of filter cake was about 21 L. The filtration took one hour.

The following table gives an overview of some of the ingredients of the provided composition, filtrate and filter cake.

TABLE 1

| | Composition | | |
|---|---|---|---|
| Value | provided | Filtrate* | Filter cake* |
| Water | 47.0 | 49.5 | 36.4 |
| Dry mass | 53.0 | 50.5 | 63.6 |
| Protein | 62.0 | 61.8 | 63.5 |
| Crude ash | 27.0 | 30.5 (+13%) | 17.6 (−35%) |
| Sulphur | 6.30 | 7.06 (+12%) | 4.24 (−33%) |
| Sodium | 7.70 | 8.65 (+12%) | 4.98 (−35%) |
| Chloride | 1.53 | 1.68 (+10%) | 1.01 (−34%) |
| Potassium | 1.12 | 1.25 (+12%) | 0.70 (−38%) |
| Phosphorus | 0.95 | 1.05 (+11%) | 0.65 (−32%) |

All data in mass percent. For moisture and dry mass, values are based on the total amount of composition used. All other values are based on the dry mass.
*In parentheses, the change compared to the provided composition in percent.

As can be seen from Table 1, the protein content hardly changes during filtration. Both in the composition used and in the filtrate as well as in the filter cake, about 62 weight percent of protein is contained. However, the proportion of crude ash in the filter cake is reduced by about one third. The mass fraction of all measured elements is reduced to the same extent. The fact that the protein content is only slightly increased indicates that not proteins but other organic substances have accumulated in the filter cake. It is assumed that these are sugar side chains of the proteins of the intestinal mucosa.

With 17 weight percent of crude ash content and about 4 and 5 weight percent of sulphur and sodium content respectively, the filter cake is surprisingly directly suitable as a feed additive.

### Example 2: Continuous Crystallisation

Filtrate obtained from Example 1 was used for the continuous crystallisation. The composition of the filtrate used is given in Table 2 below. 206.9 kg of the filtrate obtained in Example 1 was placed in a container and transferred to a freeze concentration unit (W6-9 Prodias from GEA Niro PT B.V.) for crystallisation. The precipitate was separated by centrifugation and the liquid phase (mother liquor) was obtained as product. Within 17 hours, 156 kg liquid phase and 34.9 kg precipitate were obtained. This results in a yield of 16.9 mass percent in relation to the precipitate obtained. 15.8 kg of the filtrate used was lost in the apparatus, by sticking to the apparatus or by dead spaces and the like. The temperature of the filtrate used was measured continuously to determine the crystallisation temperature. The average crystallisation temperature was −1.5° C. The temperature of the freeze concentration unit varied between 0° C. and −6° C. The viscosity of the filtrate thus obtained at a temperature of −1.25° ° C. was 70 cST (84 cP @1200 kg/m³), measured with an Ostwald viscometer. The particle size of the precipitate showed a distribution where 50% of the particles were over 100 μm in diameter. Various samples were taken throughout the process and the values of a representative sample are shown in Table 2.

TABLE 2

| | Filtrate from | | |
|---|---|---|---|
| Value | Example 1 | Liquid phase* | Precipitate* |
| Water | 48.2 | 48.5 | 54.5 |
| Dry mass | 51.8 | 51.5 | 45.5 |
| Protein | 63.9 | 75.8 (+19%) | 13.4 (−79%) |
| Crude ash | 31.2 | 14.3 (−54%) | 85.3 (+173%) |
| Sulphur | 6.73 | 3.89 (−42%) | 18.6 (+176%) |
| Sodium | 8.24 | 3.90 (−53%) | 26.59 (+223%) |
| Chloride | 1.62 | 2.07 | 0.37 |
| Potassium | 1.22 | 1.57 | 0.29 |
| Phosphorus | 1.02 | 1.25 | 0.26 |

All data in mass percent. For moisture and dry mass, values are based on the total amount of composition used. All other values are based on the dry mass.
*In parentheses, the change compared to the provided composition in percent.

While the protein content is increased in the liquid phase, the crude ash content is reduced by more than 50% in relation to the filtrate used. The liquid phase can be used as feed in this form. The precipitate has a high content of sodium and sulphur, from which it can be assumed that it is largely sodium sulphate.

### Example 3: Discontinuous Crystallisation According to Step (3)

A glass vessel equipped with a stirrer and thermometer was filled with a composition to be used. This composition is a filtrate obtained by microfiltration according to step (2) of the method of the invention. The exact composition can be found in the column of Table 3 headed “Filtrate”. The glass vessel was placed in a refrigerated chamber cooled to −10° C. The composition was stirred continuously. The experiment was terminated when the contents of the glass vessel had reached a temperature of −2.5° C. The separation of liquid phase (supernatant) and crystals was done by filtration through a Büchner funnel. The precipitate remaining in the Büchner funnel was then additionally freed from further adhering liquid phase with a hand centrifuge. The two fractions of the liquid phase were combined. The following table 3 gives an overview of some of the ingredients of the filtrate used and the liquid phase thus obtained.

TABLE 3

| Value | Filtrate | Liquid phase* |
|---|---|---|
| Protein | 57.6 | 61.3 |
| Crude ash | 33.0 | 11.7 (−64%) |
| Sulphur | 7.11 | 3.64 (−49%) |
| Sodium | 8.42 | 2.84 (−66%) |
| Chloride | 0.17 | 0.19 |
| Potassium | 0.77 | 1.16 |
| Phosphorus | 0.73 | 1.00 |

All data in mass percent based on dry mass.
*In parentheses, the change compared to the filtrate in percent.

Crystallisation and removal of the precipitate by centrifugation removes a significant proportion of the inorganic components from the filtrate obtained in step (2). As can be seen in Table 3, mainly sodium and sulphur are removed, indicating crystallisation of mainly sodium sulphate.

With a crude ash content of only 11.7 weight percent, 3.64 weight percent sulphur and 2.84 weight percent sodium, the filtrate can be used directly, for example as a feed additive.

An experiment carried out under exactly the same conditions with a non-filtered composition according to step (1) of the method of the invention did not produce any precipitate. This was also not the case when cooling down to −10° C. and when a longer test duration was used. A wide variety of compositions were used in these experiments, including the composition used in Example 1. In no case could a precipitate be obtained.

### BRIEF DESCRIPTION OF THE DRAWINGS

An exemplary embodiment of the invention is explained in greater detail below with reference to a drawing.

The drawing shows:

FIG. 1: a schematic representation of the method.

### DETAILED DESCRIPTION

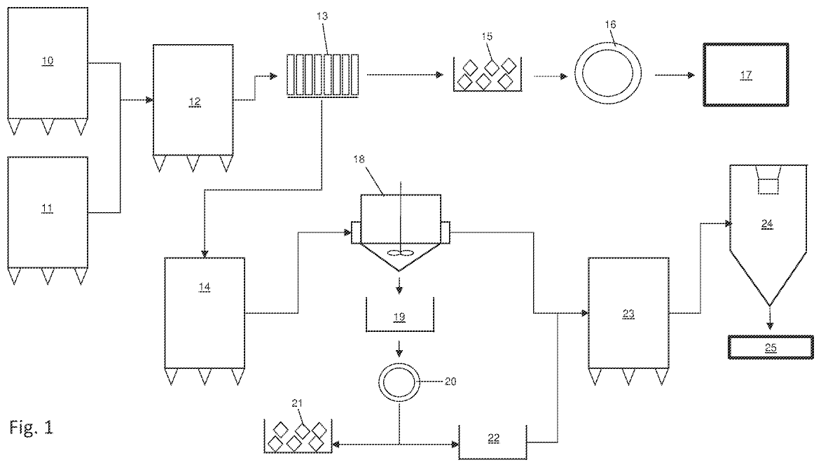

FIG. 1 shows an overview of the method for obtaining peptones from intestinal mucosa. A crude solution 10 is diluted with water 11, if necessary, to give a prepared composition 12. The prepared composition 12 is filtered through a chamber filter press 13 to give a filtrate 14 and a filter cake 15. The filter cake 15 is dried in a roller dryer 16 and a dried filter cake 17 is obtained.

The filtrate 14 obtained is cooled to an average temperature of approx. −5° C. in a freeze crystalliser 18, thus forming a precipitate 19 which is separated from the liquid phase 23. The precipitate 19 is centrifuged via a centrifuge 20 and the supernatant 22 is combined with the remaining liquid phase 23, while the precipitated salts 21 are dried.

The liquid phase 23 is dried by a spray drying unit 24 to obtain a peptone in powder form 25.

While the present disclosure has been illustrated and described with respect to a particular embodiment thereof, it should be appreciated by those of ordinary skill in the art that various modifications to this disclosure may be made without departing from the spirit and scope of the present disclosure.

What is claimed is:

1. A method comprising the following steps:
(1) providing a composition containing proteins and amino acids from mucous membranes of animals for slaughter, inorganic salts and water, wherein the sum of the mass of proteins and amino acids in the dry mass of said composition is 30 to 70 weight percent and the mass of inorganic salts in the dry mass of said composition is at least 7.5 weight percent;
(2) filtering the composition to obtain a filtrate and a filter cake;
(3) cooling the filtrate to a temperature in the range of −15° C. to 15° C. to form a precipitate;
(4) separating the resulting precipitate to obtain the liquid phase as the product.

2. The method according to claim 1, wherein the composition provided in step contains proteins and amino acids from the intestinal mucosa of animals for slaughter.

3. The method according to claim 1, wherein the composition provided in step (1) contains proteins and amino acids which are waste products of the extraction of heparin from the intestinal mucosa of animals for slaughter.

4. The method according to claim 1, wherein a microfiltration unit or a chamber filter press is used for filtering the composition in step (2).

5. The method according to claim 1, wherein the composition provided in step (1) comprises 25 to 50 weight percent of proteins and amino acids, 30 to 60, preferably 35 to 50 weight percent of water and/or 7.5 to 30 weight percent of inorganic salts.

6. The method according to claim 1, wherein a filter having a pore size greater than 2 μm is used to filter the composition in step (2).

7. The method according to claim 1, wherein the filtrate obtained in step (2) is cooled to a temperature in the range of from 12.5° C. to 12° C., preferably from 10° ° C. to 10° C. and particularly preferably from 5° C. to 5° C., to form the precipitate in step (3).

8. The method according to claim 1, wherein at least 50 weight percent of the precipitate obtained in step (4) consists of inorganic salts of the elements sodium and sulphur and also chlorine.

9. The method according to claim 1, wherein the separation of the precipitate formed in step (3) is carried out by centrifugation in step (4).

10. The method according to claim 1, wherein the liquid phase obtained in step (4) contains less than 25 weight percent of inorganic salts, particularly preferably less than 20 weight percent of inorganic salts, very particularly preferably less than 17.5 weight percent of inorganic salts, based on the dry mass.

11. The method according to claim 1, wherein the filter cake obtained in step (2) contains less than 25 weight percent of inorganic salts, particularly preferably less than 20 weight percent of inorganic salts, based on the dry mass.

12. The method according to claim 1, wherein the proportion of inorganic salts in the dry mass of the liquid phase obtained in step (4) and/or in the dry mass of the filter cake obtained in step (2) is reduced by at least 25%, preferably by at least 35% and most preferably by at least 40%, with respect to the proportion of inorganic salts in the dry mass of the composition used.

13. A peptone, wherein said peptone has been produced by a method comprising the following steps:
(1) providing a composition containing proteins and amino acids from mucous membranes of animals for slaughter, inorganic salts and water, wherein the sum of the mass of proteins and amino acids in the dry mass of said composition is 30 to 70 weight percent and the mass of inorganic salts in the dry mass of said composition is at least 7.5 weight percent;
(2) filtering the composition to obtain a filtrate and a filter cake;
(3) cooling the filtrate to a temperature in the range of −15° ° C. to 15° C. to form a precipitate;
(4) separating the resulting precipitate to obtain the liquid phase as the product.

14. A filter cake, wherein said filter cake has been produced by a method comprising the following steps:
(1) providing a composition containing proteins and amino acids from mucous membranes of animals for slaughter, inorganic salts and water, wherein the sum of the mass of proteins and amino acids in the dry mass of said composition is 30 to 70 weight percent and the mass of inorganic salts in the dry mass of said composition is at least 7.5 weight percent;
(2) filtering the composition to obtain a filtrate and a filter cake;
(3) cooling the filtrate to a temperature in the range of −15° C. to 15° C. to form a precipitate;
(4) separating the resulting precipitate to obtain the liquid phase as the product.
